SEQUENCE LISTING (8 sequences)

SEQ ID NO: 1 - DNA, 20 bp, Artificial
  primer
gacgatgtcactggctgagc

SEQ ID NO: 2 - DNA, 19 bp, Artificial
  primer
agccgccgacgctaataca

SEQ ID NO: 3 - DNA, 22 bp, Artificial
  primer
gcgacctggttaactacaatcc

SEQ ID NO: 4 - DNA, 21 bp, Artificial
  primer
cggtagtattgcccttaagcc

SEQ ID NO: 5 - DNA, 20 bp, Artificial
  primer
gctggagaaaagcagcggag

SEQ ID NO: 6 - DNA, 20 bp, Artificial
  primer
gtaagctgacgcaacgtctg

SEQ ID NO: 7 - DNA, 21 bp, Artificial
  primer
cgctttgccatgtgcagcacc

SEQ ID NO: 8 - DNA, 18 bp, Artificial
  primer
gctcagtacgatcgagcc

(19) United States
(12) Patent Application Publication
(10) Pub. No.: US 2007/0248954 A1
(43) Pub. Date: Oct. 25, 2007

(54) Primers for Use in Detecting Beta-Lactamases

(75) Inventor: Nancy D. Hanson, Gretna, NE (US)

Correspondence Address: MUETING, RAASCH & GEBHARDT, P.A., P.O. BOX 581415, MINNEAPOLIS, MN, 55458, US

(73) Assignee: Creighton University, Omaha, NE (US)

(21) Appl. No.: 10/571,027

(22) PCT Filed: Sep. 10, 2004
(86) PCT No.: PCT/US04/29695

§ 371 Date: Feb. 12, 2007

Related U.S. Application Data
(60) Provisional application No. 60/502,091, filed on Sep. 10, 2003.

(51) Int. Cl. C12Q 1/68; C07H 21/04

(52) U.S. Cl. 435/6; 536/24.33

(57) ABSTRACT

Oliognucleotide primers are provided that are specific for nucleic acid characteristic of certain β-lactamase genes. The primers can be employed in methods to identify nucleic acid characteristic of family-specific (and even group-specific) β-lactamase enzymes in samples, and particularly, in clinical isolates of Gram-negative bacteria.

### RELATED APPLICATIONS

This application claims the benefit of U.S. Provisional Application Ser. No. 60/502,091, filed 10 Sep. 2003, and U.S. Provisional Application Ser. No. 60/502,885, filed 12 Sep. 2003, each of which is incorporated herein by reference in its entirety.

### BACKGROUND

A disturbing consequence of the use, and over-use, of β-lactam antibiotics (e.g., penicillins and cephalosporins) has been the development and spread of β-lactamases. β-lactamases are enzymes that open the β-lactam ring of penicillins, cephalosporins, and related compounds, to inactivate the antibiotic. The production of β-lactamases is an important mechanism of resistance to β-lactam antibiotics among Gram-negative bacteria.

Expanded-spectrum cephalosporins have been specifically designed to resist degradation by the older broad-spectrum β-lactamases such as TEM-1, 2, and SHV-1. Microbial response to the expanded-spectrum cephalosporins has been the production of mutant forms of the older β-lactamases called extended-spectrum β-lactamases (ESBLs). Although ESBL-producing Enterobacteriaceae were first reported in Europe in 1983 and 1984, ESBLs have now been found in organisms of diverse genera recovered from patients in all continents except Antarctica. The occurrence of ESBL-producing organisms varies widely with some types more prevalent in Europe (TEM-3), others more prevalent in the United States (TEM-10, TEM-12 and TEM-26), while others appear worldwide (SHV-2 and SHV-5).

Additionally, CTX-M β-lactamases are spreading throughout North America and have been found in a wide variety of isolates within the family Enterobacteriaceae. Organisms producing CTX-M β-lactamases are typically resistant to cefotaxime, have lower minimum inhibitory concentration (MIC) values to ceftazidime and elevated MIC values to cefepime. However, these enzymes are capable of hydrolyzing the newer cephalosporins and aztreonam. Further, it is of concern that the National Committee for clinical Laboratory Standards (NCCLS) guidelines for ESBL detection in E. coli and Klebsiella spp. include an initial screening with either cefpodoxime, cefotaxime, ceftazidime, ceftriaxone, or_aztreonam followed by a confirmation test using both cefotaxime and ceftazidime in combination with clavulanate (NCCLS performance standards for antimicrobial and susceptibility testing; 12^{th} informational supplement, M100-S12 (2002)), and that a practice among some clinical laboratories is to use ceftazidime as the initial screening drug and ceftazidime with clavulanate as the confirmation test (Brenwald et al., J. Antimicrob. Chemother., 51:195-196 (2003); Dandekar et al., Diagn. Microbiol, Infect. Dis., 49:37-39 (2004)). One study has shown, however, that about 14% of ESBL-producing strains will not be detected if ceftazidime is used as an initial screen, and only about 35% of ESBL-producing strains were reported as_ESBL positive when ceftazidime with clavulanate was the only confirmation test.

It is of further concern that genes encoding β-lactamases are often located on large plasmids that also contain genes for resistance to other antibiotic classes including aminoglycosides, tetracycline, sulfonamides, trimethoprim, and chloramphenicol. Furthermore, there is an increasing tendency for pathogens to produce multiple β-lactamases. These developments, which occur over a wide range of Gram-negative genera, represent a recent evolutionary development in which common Gram-negative pathogens are availing themselves of increasingly complex repertoires of antibiotic resistance mechanisms. Clinically, this increases the difficulty of identifying effective therapies for infected patients.

Thus, there is a need for techniques that can quickly and accurately identify the types of β-lactamases that may be present in a clinical isolate or sample, for example. Surveillance studies of this nature could have significant implications in the choice of antibiotic used in hospital settings and could impact the treatment of a bacterial infection.

### SUMMARY OF THE INVENTION

The present invention is directed to the use of oligonucleotide primers specific to nucleic acids characteristic of (typically, genes encoding) certain β-lactamases. More specifically, the present invention uses primers of the invention to identify family-specific β-lactamase nucleic acids (typically, genes) in samples, particularly, in clinical isolates of Gram-negative bacteria. Even more specifically, the present invention provides primers to specifically identify groups within the CTX-M β-lactamase family.

In one aspect, the present invention is directed to a primer selected from the group of: 5′-GAC GAT GTC ACT GGC TGA GC-3′(SEQ ID NO:1); 5′-AGC CGC CGA CGC TAA TAC A-3′(SEQ ID NO:2); 5′-GCG ACC TGG TTA ACT ACA ATC C-3′(SEQ ID NO:3); 5′-CGG TAG TAT TGC CCT TAA GCC-3′(SEQ ID NO:4); 5′-GCT GGA GAA AAG CAG CGG AG-3′(SEQ ID NO:5); 5′-GTA AGC TGA CGC AAC GTC TG-3′(SEQ ID NO:6); 5′-CGC TTT GCC ATG TGC AGC ACC-3′(SEQ ID NO:7); and 5′-GCT CAG TAC GAT CGA GCC-3′(SEQ ID NO:8); and full-length complements thereof.

In a further aspect, the present invention is directed to a primer pair including one primer selected from the group of: 5′-GAC GAT GTC ACT GGC TGA GC-3′(SEQ ID NO:1); 5′-AGC CGC CGA CGC TAA TAC A-3′(SEQ ID NO:2); 5′-GCG ACC TGG TTA ACT ACA ATC C-3′(SEQ ID NO:3); 5′-CGG TAG TAT TGC CCT TAA GCC-3′(SEQ ID NO:4); 5′-GCT GGA GAA AAG CAG CGG AG-3′(SEQ ID NO:5); 5′-GTA AGC TGA CGC AAC GTC TG-3′(SEQ ID NO:6); 5′-CGC TTT GCC ATG TGC AGC ACC-3′(SEQ ID NO:7); and 5′-GCT CAG TAC GAT CGA GCC-3′(SEQ ID NO:8); and full-length complements thereof.

In yet further aspects, the present invention is directed to a primer selected from the group of 5′-GAC GAT GTC ACT GGC TGA GC-3′(SEQ ID NO:1); 5′-AGC CGC CGA CGC TAA TAC A-3′(SEQ ID NO:2); and full-length complements thereof; a primer selected from the group of 5′-GCG ACC TGG TTA ACT ACA ATC C-3′(SEQ ID NO:3); 5′-CGG TAG TAT TGC CCT TAA GCC-3′(SEQ ID NO:4); and full-length complements thereof; a primer selected from the group of 5′-GCT GGA GAA AAG CAG CGG AG-3′(SEQ ID NO:5); 5′-GTA AGC TGA CGC AAC GTC TG-3′(SEQ ID NO:6); and full-length complements thereof; and to a primer selected from the group of 5′-CGC TTT GCC ATG TGC AGC ACC-3′(SEQ ID NO:7); and 5′-GCT CAG TAC GAT CGA GCC-3′(SEQ ID NO:8); and full-length complements thereof.

As used herein, a nucleic acid characteristic of a β-lactamase enzyme includes a gene or a portion thereof. A “gene” as used herein, is a segment or fragment of nucleic acid (e.g., a DNA molecule) involved in producing a peptide (e.g., a polypeptide and/or protein). A gene can include regions preceding (upstream) and following (downstream) a coding region (i.e., regulatory elements) as well as intervening sequences (introns, e.g., non-coding regions) between individual coding segments (exons). The term “coding region” is used broadly herein to mean a region capable of being transcribed to form an RNA, the transcribed RNA can be, but need not necessarily be, further processed to yield an mRNA.

Additionally, a method for identifying a β-lactamase in a clinical sample is provided. Preferably, the clinical sample provided is characterized as Gram-negative bacteria with resistance to β-lactam antibiotics. In one aspect, the method of the present invention for identifying a β-lactamase in a clinical sample includes, providing a pair of oligonucleotide primers specific for one or more groups within the CTX-M β-lactamase family, wherein one primer of the pair is complementary to at least a portion of the β-lactamase nucleic acid in the sense strand and the other primer of each pair is complementary to at least a portion of the β-lactamase nucleic acid in the antisense strand; annealing the primers to the β-lactamase nucleic acid; simultaneously extending the annealed primers from a 3′ terminus of each primer to synthesize an extension product that is complementary to the nucleic acid strands annealed to each primer wherein each extension product after separation from the β-lactamase nucleic acid serves as a template for the synthesis of an extension product for the other primer of each pair; separating the amplified products; and analyzing the separated amplified products for a region characteristic of the β-lactamase. As discussed below, the CTX-M β-lactamase family includes several groups of β-lactamases within the family. The present invention provides a method for identifying one or more groups within the CTX-M β-lactamase family.

The method, described above, employs oligonucleotide primer pairs that are specific for one or more groups within the CTX-M family of β-lactamases, particularly primer pairs specific for one or more groups of CTX-M β-lactamases such as the β-lactamases of Group 1: CTX-M-1, 3, 10-12, 15 (also known as UOE-1), 22, 23, 28, 29, and 30; Group 2: CTX-M-2, 4-7, 20, and TOHO-1, Group 3: CTX-M-8; and Group 4: CTX-M-9, 13, 14, 16-19, 21, 27, and TOHO-2. The primers may also be specific for nucleic acid of the β-lactamases of Group 5: CTX-M-25 and 26. The primer pairs may be specific for one group within the CTX-M β-lactamase family or more than one group within the CTX-M β-lactamase family (e.g., a primer pair specific for two groups within the CTX-M β-lactamase family); however, none of the primer pairs of the present invention are specific for all CTX-M β-lactamase family groups (in which case they would be only family-specific but not group-specific). That is, although the primer pairs of the present invention can distinguish a CTX-M β-lactamase from another β-lactamase family (e.g., a TEM, SHV, or OXA family), the primer pairs of the present invention can also distinguish between different groups within the CTX-M β-lactamase family.

Real-time polymerase chain reaction (PCR) is recognized in the art as a useful tool that may provide advantages over traditional PCR, as described more thoroughly below. Thus, in yet another aspect, the present invention is also directed to a method for identifying a β-lactamase in a clinical sample, the method including:

providing a primer pair comprising one primer selected from the group of: 5′-GAC GAT GTC ACT GGC TGA GC-3′(SEQ ID NO:1); 5′-AGC CGC CGA CGC TAA TAC A-3′(SEQ ID NO:2); 5′-GCG ACC TGG TTA ACT ACA ATC C-3′(SEQ ID NO:3); 5′-CGG TAG TAT TGC CCT TAA GCC-3′(SEQ ID NO:4); 5′-GCT GGA GAA AAG CAG CGG AG-3′(SEQ ID NO:5); 5′-GTA AGC TGA CGC AAC GTC TG-3′(SEQ ID NO:6); 5′-CGC TTT GCC ATG TGC AGC ACC-3′(SEQ ID NO:7); and 5′-GCT CAG TAC GAT CGA GCC-3′(SEQ ID NO:8); and full-length complements thereof; and subjecting the primer pair to a real-time polymerase chain reaction assay.

The present invention further provides kits useful in detecting a β-lactamase of interest in a clinical sample. In one aspect, the present invention provides a diagnostic kit for detecting a CTX-M β-lactamase which includes packaging, containing, separately packaged: at least one primer pair capable of hybridizing to a β-lactamase nucleic acid selected from the group of members of Groups 1-5 of the CTX-M β-lactamase family; a positive and negative control; and a protocol for identification of the β-lactamase nucleic acid of interest, wherein the at least one primer pair is specific for one or more groups within the CTX-M β-lactamase family.

In a further aspect, the present invention is directed to a diagnostic kit for detecting a family of CTX-M β-lactamase which includes packaging, containing, separately packaged: at least one primer pair capable of hybridizing to a β-lactamase nucleic acid of interest; a positive and negative control; and a protocol for identification of the β-lactamase nucleic acid of interest; wherein the primers are selected from the group of: 5′-GAC GAT GTC ACT GGC TGA GC-3′(SEQ ID NO:1); 5′-AGC CGC CGA CGC TAA TAC A-3′(SEQ ID NO:2); 5′-GCG ACC TGG TTA ACT ACA ATC C-3′(SEQ ID NO:3); 5′-CGG TAG TAT TGC CCT TAA GCC-3′(SEQ ID NO:4); 5′-GCT GGA GAA AAG CAG CGG AG-3′(SEQ ID NO:5); 5′-GTA AGC TGA CGC AAC GTC TG-3′(SEQ ID NO:6); 5′-CGC TTT GCC ATG TGC AGC ACC-3′(SEQ ID NO:7); and 5′-GCT CAG TAC GAT CGA GCC-3′(SEQ ID NO:8); and full-length complements thereof.

Additionally, in yet another aspect, the present invention is also directed to a diagnostic kit for detecting a family of CTX-M β-lactamase using real time polymerase chain reaction which includes packaging, containing, separately packaged: at least one primer pair capable of hybridizing to a β-lactamase nucleic acid of interest; a positive and negative control; and a protocol for identification of the β-lactamase nucleic acid of interest; wherein one primer of the pair of primers is selected from the group of: 5′-GAC GAT GTC ACT GGC TGA GC-3′(SEQ ID NO:1); 5′-AGC CGC CGA CGC TAA TAC A-3′(SEQ ID NO:2); 5′-GCG ACC TGG TTA ACT ACA ATC C-3′(SEQ ID NO:3); 5′-CGG TAG TAT TGC CCT TAA GCC-3′(SEQ ID NO:4); 5′-GCT GGA GAA AAG CAG CGG AG-3′(SEQ ID NO:5); 5′-GTA AGC TGA CGC AAC GTC TG-3′(SEQ ID NO:6);

5′-CGC TTT GCC ATG TGC AGC ACC-3′(SEQ ID NO:7); and 5′-GCT CAG TAC GAT CGA GCC-3′(SEQ ID NO:8); and full-length complements thereof.

### BRIEF DESCRIPTION OF THE DRAWINGS

FIGS. 1-2 are primers of the present invention.

FIG. 3 is a CTX-M PCR of four primer sets representing groups of bla_{CTXM} genes.

### DETAILED DESCRIPTION OF CERTAIN EMBODIMENTS

The present invention is directed to the detection of nucleic acid that is characteristic of (e.g., at least a segment of a gene that codes for) family-specific β-lactamase nucleic acid in samples (e.g., clinical isolates of Gram-negative bacteria). Specifically, the present invention is directed to the detection of β-lactamase nucleic acid (preferably, a gene or at least a segment of a gene) using unique primers and the polymerase chain reaction (PCR). Using the primers and methods of the present invention, β-lactamases belonging to the CTX-M family (e.g., β-lactamases of Groups 1-5 as indicated above), for example, can be identified.

Even more specifically, the present invention provides primers to specifically identify groups within the CTX-M β-lactamase family. Accordingly, using the primers and methods of the present invention, not only can β-lactamases belonging to the CTX-M family be distinguished from other β-lactamases, but various groups within the CTX-M family (e.g., β-lactamases of Groups 1-5 as indicated herein) can be identified. This does not mean, however, that any one primer pair must be specific for only one group within the family, although this is preferred. Certain primers are specific for two (or more), but not all, groups, for example, which is still advantageous because it allows for narrowing the group identification.

PCR amplification and sequencing of genes for use in characterizing organisms producing CTX-M-β-lactamases is known in the art, such as, for example, the molecular approach to screening ESBL-positive organisms for the presence of CTX-M genes as described in Edelstein et al. (Antimicrob. Agents Chemother., 47:3724-3732 (2003)). In this approach, consensus primers (i.e., “universal” primers), which recognize all known variant genes of bla_{CTX-M} known in the art at the time, are used to generate an amplified product of 544 base pairs. Following this reaction, the specific groups of bla_{CTX-M} genes are identified by the use of restriction fragment length polymorphism (RFLP) analysis. The present invention provides an advantage over the above approach in that specific primer sets are used to detect various CTX-M-β-lactamase genes and/or groups of CTX-M-β-lactamase genes, negating the need to perform an additional identification step (at least for most embodiments). That is, it should be understood that for most embodiments of the present invention, an additional identification step is not necessarily needed, but one could be used if desired, particularly if the primers are specific for two or more groups and there is a desire to distinguish between these groups or to identify the specific CTX-M β-lactamase family member.

The present invention provides amplification of a single DNA fragment for a CTX-M-β-lactamase gene, affording simple interpretation of results that can be adapted for use in reference laboratories for screening multiple isolates for the presence of CTX-M-β-lactamase genes. For example, to identify a β-lactamase of interest contained in a sample, such as CTX-M-14 β-lactamase, a selected primer pair of the invention (e.g., SEQ ID NO. 5 and SEQ ID NO. 6 for CTX-M-14 β-lactamase) is used to provide an amplified product, which, following a gel electrophoresis assay of the amplified products, provides identification of the β-lactamase of interest (i.e., the CTX-M-14 β-lactamase) in a single step. That is, no additional identification steps, such as RFLP, WAVE analysis, sequence identification (Gold Standard), or single stranded conformational polymorphisms (SSCP), are required to identify the β-lactamases of interest, although such additional assays could be performed, if desired. Thus, the present invention is distinguished over the methods of Edelstein et al. as the Edelstein et al. methods require the use of the additional RFLP identification step and may require yet another identification step beyond the RFLP analysis.

Additionally, the step of analyzing the separated amplified products, as recited in the present claims, includes a visual inspection of the gel produced by gel electrophoresis of the amplified products. The gel produced by the present methods typically provides a clear, unambiguous band; thus, the primers and methods of the present invention may be performed by personnel with lesser training and/or experience and still provide accurate results. The methods of Edelstein et al., however, typically do not produce as clear a product as do the methods of the present invention, as the results of RFLP analysis are often difficult to interpret and do not give the single band results provided by the primers and methods of the present_invention. Thus, the methods of Edelstein et al. generally provide more ambiguous results, which are typically more difficult for an untrained person to correctly interpret.

Further, the methods of the present invention provide primer pairs specific for one or more groups within the CTX-M β-lactamase family, which is defined herein to include primer pairs specific for one group (e.g., β-lactamases found in Group 1 of the CTX-M family) and also primer pairs specific for more than one group (e.g., β-lactamases found in Groups 3 and 5 of the CTX-M family); however, the primer pairs are not specific for all β-lactamase family groups. That is, the present invention provides a method for identifying in a single step a β-lactamase included in the CTX-M β-lactamase family over a β-lactamase included in another family of β-lactamases (e.g., an OXA family), as well as provides a method of distinguishing one group of the CTX-M β-lactamase family over another group of the CTX-M β-lactamase family. This is distinguished over the methods of Edelstein et al., for example, which do not distinguish, and in particular do not distinguish in one step, between groups within the CTX-M β-lactamase family. Edelstein et al. provide a method whereby universal primers are used with PCR to provide amplified products, which may include all CTX-M β-lactamases from other β-lactamases. Further identification is then required to determine the β-lactamase in the sample. The present invention, advantageously, provides the ability to identify in a clinical sample, in a single step, the presence of one group over another group of the CTX-M β-lactamase family.

The methods of the present invention involve the use of the polymerase chain reaction sequence amplification method (PCR) using novel primers. U.S. Pat. No. 4,683,195 (Mullis et al.) describes a process for amplifying, detecting, and/or cloning nucleic acid. Preferably, this amplification method relates to the treatment of a sample containing nucleic acid (typically, DNA) of interest from bacteria, particularly Gram-negative bacteria, with a molar excess of an oligonucleotide primer pair, heating the sample containing the nucleic acid of interest to yield two single-stranded complementary nucleic acid strands, adding the primer pair to the sample containing the nucleic acid strands, allowing each primer to anneal to a particular strand under appropriate temperature conditions that permit hybridization, providing a molar excess of nucleotide triphosphates and polymerase to extend each primer to form a complementary extension product that can be employed in amplification of a desired nucleic acid, detecting the amplified nucleic acid, and analyzing the amplified nucleic acid for a size specific amplicon (as indicated below) characteristic of the specific β-lactamase of interest. This process of heating, annealing, and synthesizing is repeated many times, and with each cycle the desired nucleic acid increases in abundance. Within a short period of time, it is possible to obtain a specific nucleic acid, e.g., a DNA molecule, that can be readily purified and identified.

The oligonucleotide primer pair, which may include at least one primer selected from the group of SEQ ID NO. 1 to SEQ ID NO. 8, includes one primer that is substantially complementary to at least a portion of a sense strand of the nucleic acid and one primer that is substantially complementary to at least a portion of an antisense strand of the nucleic acid. The process of forming extension products preferably involves simultaneously extending the annealed primers from a 3′ terminus of each primer to synthesize an extension product that is complementary to the nucleic acid strands annealed to each primer wherein each extension product after separation from the β-lactamase nucleic acid serves as a template for the synthesis of an extension product for the other primer of each pair. The amplified products are preferably detected by size fractionization using gel electrophoresis. Variations of the method are described in U.S. Pat. No. 4,683,194 (Saiki et al.). The polymerase chain reaction sequence amplification method is also described by Saiki et al., Science, 230, 1350-1354 (1985) and Scharf et al., Science, 324, 163-166 (1986).

An “oligonucleotide,” as used herein, refers to a polymeric form of nucleotides of any length, either ribonucleotides or deoxyribonucleotides. The term oligonucleotide refers particularly to the primary structure, and thus includes double and single-stranded DNA molecules and double and single-stranded RNA molecules.

A “primer,” as used herein, is an oligonucleotide that is complementary to at least a portion of nucleic acid of interest and, after hybridization to the nucleic acid, may serve as a starting-point for the polymerase chain reaction. The terms “primer” or “oligonucleotide primer,” as used herein, further refer to a primer, having a nucleotide sequence that possesses a high degree of nucleic acid sequence similarity to at least a portion of the nucleic acid of interest. “High degree” of sequence similarity refers to a primer that typically has at least about 80% nucleic acid sequence similarity, and preferably about 90% nucleic acid sequence similarity. Sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

The terms “complement” and “complementary” as used herein, refer to a nucleic acid that is capable of hybridizing to a specified nucleic acid molecule under stringent hybridization conditions. Stringent hybridization conditions include, for example, temperatures from about 50° C. to about 65° C., and magnesium (Mg) concentrations from about 1.5 millimolar (mM) to about 2.0 mM. Thus, a specified DNA molecule is typically “complementary” to a nucleic acid if hybridization occurs between the specified DNA molecule and the nucleic acid. “Complementary,” further refers to the capacity of purine and pyrimidine nucleotides to associate through hydrogen bonding in double stranded nucleic acid molecules. The following base pairs are complementary: guanine and cytosine; adenine and thymine; and adenine and uracil.

As used herein, the terms “amplified molecule,” “amplified fragment,” and “amplicon” refer to a nucleic acid molecule (typically, DNA) that is a copy of at least a portion of the nucleic acid and its complementary sequence. The copies correspond in nucleotide sequence to the original molecule and its complementary sequence. The amplicon can be detected and analyzed by a wide variety of methods. These include, for example, gel electrophoresis, single strand conformational polymorphism (SSCP), restriction fragment length polymorphism (RFLP), capillary zone electrophoresis (CZE), and the like. Preferably, using methods and primers of the present invention, the amplicon can be detected, and hence, the type of β-lactamase identified, using gel electrophoresis and appropriately sized markers, according to techniques known to one of skill in the art.

The primers are oligonucleotides, either synthetic or naturally occurring, capable of acting as a point of initiating synthesis of a product complementary to the region of the DNA molecule containing the β-lactamase of interest. The primer includes nucleotides capable of hybridizing under stringent conditions to at least a portion of at least one strand of a nucleic acid molecule of a β-lactamase selected from the group of CTX-M-1, CTX-M-2, CTX-M-3, CTX-M-4, CTX-M-5, CTX-M-6, CTX-M-7, CTX-M-8, CTX-M-9, CTX-M-10, CTX-M-11, CTX-M-12, CTX-M-13, CTX-M-14, CTX-M-15 (also known as UOE-1), CTX-M-16, CTX-M-17, CTX-M-18, CTX-M-l9, CTX-M-20, CTX-M-21, CTX-M-22, CTX-M-23, CTX-M-25, CTX-M-26,_CTX-M-27, CTX-M-28, CTX-M-29, CTX-M-30, TOHO-1, TOHO-2, or any combination of these β-lactamases.

Preferably, the primers of the present invention typically have at least about 15 nucleotides, preferably at least about 18 nucleotides, and more preferably at least about 20 nucleotides. Typically, the primers have no more than about 30 nucleotides, preferably no more than about 28 nucleotides, more preferably no more than about 26 nucleotides, even more preferably no more than about 24 nucleotides, and still more preferably no more than about 22 nucleotides. The primers are chosen such that they preferably produce a primed product of at least about 300 base pairs and preferably no greater than about 600 base pairs, more preferably no greater than about 500 base pairs.

Optionally, a primer used in accordance with the present invention includes a label constituent. The label constituent can be selected from the group of an isotopic label, a fluorescent label, a polypeptide label, and a dye release compound. The label constituent is typically incorporated in the primer by including a nucleotide having the label attached thereto. Isotopic labels preferably include those compounds that are beta, gamma, or alpha emitters, more preferably isotopic labels are selected from the group of ³²P, ³⁵S, and ¹²⁵I. Fluorescent labels are typically dye compounds that emit visible radiation in passing from a higher to a lower electronic state, typically in which the time interval between adsorption and emission of energy is relatively short, generally on the order of about 10⁻⁸ to about 10⁻³ second. Suitable fluorescent compounds that can be utilized include fluorescien and rhodamine, for example. Suitable polypeptide labels that can be utilized in accordance with the present invention include antigens (e.g., biotin, digoxigenin, and the like) and enzymes (e.g., horse radish peroxidase). A dye release compound typically includes chemiluminescent systems defined as the emission of absorbed energy (typically as light) due to a chemical reaction of the components of the system, including oxyluminescence in which light is produced by chemical reactions involving oxygen.

While PCR, through the use of repetitive multiplication of template molecules, is a sensitive and extremely useful analytical tool, such repetitive multiplication also provides a drawback in that small differences in the template can result in significant differences in the amount of product. One answer to this concern is the use of “real-time” PCR. Thus, in addition to the use of PCR described above (i.e., “traditional” PCR), primers, methods, and kits of the present invention may also be useful in carrying out real-time PCR.

Real-time PCR monitors the fluorescence emitted during the PCR reaction as an indicator of amplicon production during the PCR cycle (i.e., in real-time) to provide data collected in the exponential phase of the reaction. Traditional PCR, on the other hand, provides data on the amount of amplified product only at the end-point of the reaction.

Real-time PCR is based on the detection and quantitation of a fluorescent reporter or intercalation of a florescent dye, such as SYBR®Green into the amplified PCR product. The signal increases in direct proportion to the amount of PCR product in a reaction and, by recording the amount of fluorescence emission at each cycle, the PCR reaction may be monitored during the exponential phase. Additionally, real-time PCR substantially eliminates the need for post-PCR processing of PCR products, thus increasing throughput and decreasing the possibility of carryover contamination. Further, compared to traditional PCR, real-time PCR provides a wider dynamic range of an assay (i.e., determination of how much the target concentration can vary and still be quantified), and, thus, improved accuracy in quantitation of the PCR product. Real-time PCR may also be used for applications that would be less effective with traditional PCR (Dorak, Real-Time PCR, available on the World Wide Web at http//dorakmt.tripod.com/genetics/realtime.html, 2004).

Real-time PCR methods typically use primers that are shorter than those used in traditional PCR methods; however, smaller amplicons are generally provided, typically at least about 150 base pairs, more preferably at least about 100 base pairs, and preferably no greater than about 200 base pairs. Amplification of shorter products aids in the advantage provided by real-time PCR, as the longer the product, the longer time it takes for amplification, thus the “real-time” analysis will likely be jeopardized.

Real-time PCR may be used with primers and methods of the present invention wherein the primer pair used includes one primer selected from the primers of SEQ ID No. 1 to SEQ ID NO. 8, and another primer, resulting in an amplicon having, typically, no fewer than about 100 base pairs and no more than about 250 base pairs. Additionally, the present invention provides a kit for use with real-time PCR, the kit including a primer pair wherein one primer of the primer pair is selected from the group of SEQ ID NO. 1 to SEQ ID NO. 8.

Preferred examples of primers of the present invention specific for certain β-lactamases are as follows, wherein “F” in the designations of the primers refers to a 5′ upstream primer and “R” refers to a 340 downstream primer. For those β-lactamases that have more than one upstream primer and more than one downstream primer listed below as preferred primers, various combinations can be used. Typically, hybridization conditions utilizing primers of the invention include, for example, a hybridization temperature of at least about 50° C. and no greater than about 62° C., and a Mg concentration of at least about 1.5 mM (millimolar) and no greater than about 2.0 mM. Although lower temperatures and higher concentrations of Mg can be employed, this may result in decreased primer specificity.

The following primers are specific for nucleic acid characteristic of the CTX-M-1, 3, 10-12, 15 (UOE-1), 22, 23, 28, 29 and 30 β-lactamase enzymes.

| | | |
|---|---|---|
| Primer Name: | CTXM1-F3 | |
| Primer Sequence: | 5′-GAC GAT GTC ACT GGC TGA GC-3′ | (SEQ ID NO:1) |
| Primer Name: | CTXM1-R2 | |
| Primer Sequence: | 5′-AGC CGC CGA CGC TAA TAC A-3′ | (SEQ ID NO:2) |

Employing a primer pair containing the primer sequences of SEQ ID NO:1 and SEQ ID NO:2 to a sample containing a CTX-M-1, 3, 10-12, 15 (UOE-1), 22, 23, 28, 29 and/or 30 β-lactamase (a Group 1 CTX-M β-lactamase), a size-specific amplicon of 499 base pairs will typically be obtained.

The following primers are specific for nucleic acid characteristic of the CTX-M-2, 4, 5, 6, 7, and 20 β-lactamase enzymes, as well as TOHO-1 β-lactamase.

| | | |
|---|---|---|
| Primer Name: | TOHO 1-2F | |
| Primer Sequence: | 5′-GCG ACC TGG TTA ACT ACA ATC C-3′ | (SEQ ID NO:3) |
| Primer Name: | TOHO 1-1R | |
| Primer Sequence: | 5′-CGG TAG TAT TGC CCT TAA GCC-3′ | (SEQ ID NO:4) |

Employing a primer pair containing the primer sequences of SEQ ID NO:3 and SEQ ID NO:4 to a sample containing a CTX-M-2, 4, 5, 6, 7, and/or β-lactamase, and/or a TOHO-1 β-lactamase (a Group 2 CTX-M β-lactamase), a size-specific amplicon of 351 base pairs will typically be obtained.

The following primers are specific for nucleic acid characteristic of the CTX-M-9, 13, 14, 16, 17, 18, 19, 21, and 27 β-lactamase enzymes, as well as TOHO-2 β-lactamase.

| | | |
|---|---|---|
| Primer Name: | CTXM914F | |
| Primer Sequence: | 5′-GCT GGA GAA AAG CAG CGG AG-3′ | (SEQ ID NO:5) |
| Primer Name: | CTXM914R | |
| Primer Sequence: | 5′-GTA AGC TGA CGC AAC GTC TG-3′ | (SEQ ID NO:6) |

Employing a primer pair containing the primer sequences of SEQ ID NO:5 and SEQ ID NO:6 to a sample containing a CTX-M-9, 13, 14, 16, 17, 18, 19, 21, and/or 27 β-lactamase, and/or a TOHO-2 β-lactamase (a Group 4 CTX-M β-lactamase), a size-specific amplicon of 474 base pairs will typically be obtained.

The following primers are specific for nucleic acid characteristic of the CTX-M-8, 25, and 26 β-lactamase enzymes.

| | | |
|---|---|---|
| Primer Name: | CTXM825F | |
| Primer Sequence: | 5′-CGC TTT GCC ATG TGC AGC ACC-3′ | (SEQ ID NO:7) |
| Primer Name: | CTXM825R | |
| Primer Sequence: | 5′-GCT CAG TAC GAT CGA GCC-3′ | (SEQ ID NO:8) |

Employing a primer pair containing the primer sequences of SEQ ID NO:7 and SEQ ID NO:8 to a sample containing a CTX-M-8, 25, and/or 26 β-lactamase (a Group 3 or Group 5 CTX-M β-lactamase), a size-specific amplicon of 307 base pairs will typically be obtained.

Various other primers, or variations of the primers described above, can also be prepared and used according to methods of the present invention. For example, alternative primers can be designed based on targeted β-lactamase genes known or suspected to contain regions possessing high G/C content (i.e., the percentage of guanine and cytosine residues). As used herein, a “high G/C content” in a target nucleic acid, typically includes regions having a percentage of guanine and cytosine residues of about 60% to about 90%. Thus, changes in a prepared primer will alter, for example, the hybridization or annealing temperatures of the primer, the size of the primer employed, and the sequence of the specific resistance gene or nucleic acid to be identified. Therefore, manipulation of the G/C content, e.g., increasing or decreasing, of a primer or primer pair may be beneficial in increasing detection sensitivity in the method.

Oligonucleotides of the invention can readily be synthesized by techniques known in the art (see, for example, Crea et al., Proc. Natl. Acad. Sci. (U.S.A.) 75:5765 (1978)).

Once the primers are designed, their specificity can be tested using the following method. Depending on the target nucleic acid of clinical interest, a nucleic acid is isolated from a bacterial control strain known to express or contain the resistance gene. This control strain, as used herein, refers to a “positive control” nucleic acid (typically, DNA). Additionally, a “negative control” nucleic acid (typically, DNA) can be isolated from one or more bacterial strains known to express a resistance gene other than the target gene of interest. Using the polymerase chain reaction, the designed primers are employed in a detection method, as described above, and used in the positive and negative control samples and in at least one test sample suspected of containing the resistance gene of interest. The positive and negative controls provide an effective and qualitative (or grossly quantitative) means by which to establish the presence or the absence of the gene of interest of test clinical samples. It should be recognized that with a small percentage of primer pairs, possible cross-reactivity with other β-lactamase genes might be observed. However, the size and/or intensity of any cross-reactive amplified product will be considerably different and can therefore be readily evaluated and dismissed as a negative result.

The invention also relates to kits for identifying a family-specific β-lactamase enzyme by PCR analysis. Kits of the invention typically include one or more primer pairs specific for a β-lactamase of interest, one or more positive controls, one or more negative controls, and protocol for identification of the β-lactamase of interest using polymerase chain reaction.

Primer pairs useful in kits of the present invention include those selected from the group of SEQ ID NO. 1 to SEQ ID NO. 8. Additionally, kits useful with real-time PCR methods include primer pairs wherein one primer of the pair is selected from the group of SEQ ID No. 1 to SEQ ID NO. 8. A negative control includes a nucleic acid (typically, DNA) molecule encoding a resistant β-lactamase gene other than the β-lactamase gene of interest. The negative control nucleic acid may be a naked nucleic acid (typically, DNA) molecule or inserted into a bacterial cell. Preferably, the negative control nucleic acid is double-stranded; however, a single-stranded nucleic acid may be employed. A positive control includes a nucleic acid (typically, DNA) that encodes a β-lactamase gene from the family of β-lactamase genes of interest. The positive control nucleic acid may be a naked nucleic acid molecule or inserted into a bacterial cell, for example. Preferably, the positive control nucleic acid is double-stranded, however, a single-stranded nucleic acid may be employed. Typically, the nucleic acid is obtained from a bacterial lysate.

Accordingly, the present invention provides a kit for characterizing and identifying a family-specific β-lactamase gene that would have general applicability. Preferably, the kit includes a polymerase (typically, DNA polymerase) enzyme, such as Taq polymerase, and the like. A kit of the invention also preferably includes at least one primer pair that is specific for a β-lactamase. A buffer system compatible with the polymerase enzyme is also included and are well known in the art. Optionally, the at least one primer pair may contain a label constituent, a fluorescent label, a polypeptide label, and a dye release compound. The kit may further contain at least one internal sample control, in addition to one or more further means required for PCR analysis, such as a reaction vessel. If required, a nucleic acid from the bacterial sample can be isolated and then subjected to PCR analysis using the provided primer set of the invention.

In another embodiment, family-specific β-lactamase genes in clinical samples, particularly clinical samples containing Gram-negative bacteria, can be detected by the primers described herein in a “microchip” detection method. In a microchip detection method, nucleic acid, e.g., genes, of multiple β-lactamases in clinical samples can be detected with a minimal requirement for human intervention. Techniques borrowed from the microelectronics industry are particularly suitable to these ends. For example, micromachining and photolithographic procedures are capable of producing multiple parallel microscopic scale components on a single chip substrate. Materials can be mass produced and reproducibility is exceptional. The microscopic sizes minimize material requirements. Thus, human manipulations can be minimized by designing a microchip type surface capable of immobilizing a plurality of primers of the invention on the microchip surface.

Thus, an object of the present invention is to provide a parallel screening method wherein multiple serial reactions are automatically performed individually within one reaction well for each of the plurality of nucleic acid strands to be detected in the plural parallel sample wells. These serial reactions are performed in a simultaneous run within each of the multiple parallel lanes of the device. “Parallel” as used herein means wells identical in function. “Simultaneous” means within one preprogrammed run. The multiple reactions automatically performed within the same apparatus minimize sample manipulation and labor.

Thus, the present invention provides multiple reaction wells, the reaction wells being reaction chambers, on a microchip, each reaction well containing an individualized array to be used for detecting a β-lactamase gene uniquely specified by the substrates provided, the reaction conditions and the sequence of reactions in that well. The chip can thus be used as a method for identifying β-lactamase genes in clinical samples.

Objects and advantages of this invention are further illustrated by the following example, but the particular materials and amounts thereof recited in these examples, as well as other conditions and details, should not be construed to unduly limit this invention.

### EXAMPLE 1

### CTX-M Type-Specific Identification in Enterobacteriaceae Using CTX-M Family-Specific Primers

Methods:

Twenty two isolates representing E. coli (Ec), Citrobacter freundii (Cf) and C. koseri (Ck) were studied. All of these strains had MICs of greater than (>) 32 micrograms per milliliter (mg/ml) of cefotaxime, >16 mg/ml of cefepime, and ranged between 1 to 8 mg/ml for ceftazidime. Dendrogram analysis of CTX-M genes deposited in GenBank was performed, and based on these analyses, known CTX-M-genes were divided into four families. Based on these families, four sets of family-specific primers were designed. Specificity of the primers was tested on all 22 of the isolates.

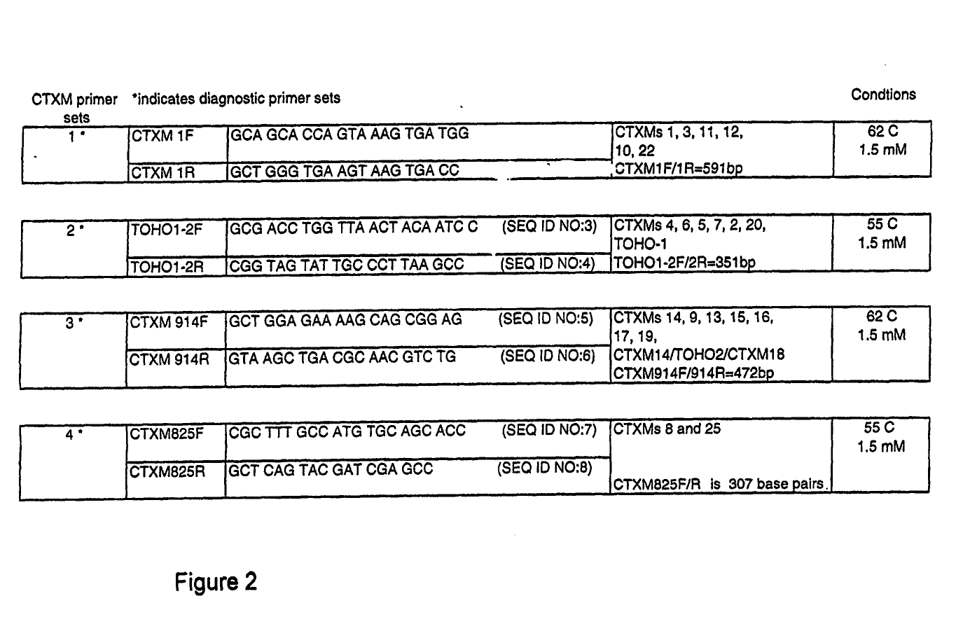

Polymerase Chain Reaction (PCR) amplifications were carried out under conditions as indicated in FIG. 2, which shows diagnostic primer sets and PCR conditions, with hybridization temperature indicated in degrees Centigrade and 1.5 millimolar (mM) MgCl₂ concentration. Selected PCR products were then sequenced to verify family specificity.

Results:

DNA from all strains was tested with every primer set. Seventy-seven percent (17/22) of the isolates were identified as carrying CTX-M genes. Of these, 68% (15/22) were E. coli, and 5% (1/22) were Citrobacter freundii. No CTX-M genes were identified in the C. koseri isolates. Of the positive CTX-M E. coli isolates, 60% (9/15) were CTX-M-14-like and 40% (6/15) were CTX-M-1-like. The Citrobacter freundii isolate carried a CTX-M-1-like gene. Sequencing of selected amplicons verified that the amplicons belonged to the CTX-M-14 or CTX-M-1 group of CTX-M genes. No amplification products were observed for the other two family-specific primer sets.

Conclusions:

Susceptibility profiles alone cannot predict CTX-M producing organisms. Twenty-three percent of isolates with similar MIC profiles were negative for CTX-M-like genes. Accurate surveillance of CTX-M producers will require susceptibility profiles combined with diagnostic PCR using family-specific primer sets.

### EXAMPLE 2

### Identification of β-lactamase Genes in Clinical Strains Producing Extended-Spectrum β-lactamases Using CTX-M Family-Specific Primers

Methods:

A total of 175 isolates representing E. coli (n=168), K. pneumoniae (n=7) were studied. Minimum inhibitory concentrations (MICs) to the following drugs were determined by Vitek (Vitek AMS; bioMérieux Vitek 5 Systems Inc., Hazelwood, Mo.); piperacillin (PIP), piperacillin/tazobactam (TZP), cefpodoxime (CPD), cefotaxime (CTX), ceftazidime (CAZ). The quality control strains used for this study were E. coli ATCC 25922, E. coli 35218, Pseudomonas aeruginosa ATCC 27853, Staphlylococcus aureus ATCC 29213, and K. pneumoniae ATCC 700603. Throughout this study, results were interpreted using National Committee for Clinical Laboratory Standards (NCCLS) criteria for broth dilution.

The presence of ESBLs was evaluated in both the control strains and the recent clinical isolates. Screening was performed with Vitek (Vitek AMS; bioMerieux Vitek Systems, Inc., Hazelwood, Mo.) using 1 μg/ml CPD, CAZ, and CTX. Screening and disk confirmation tests using CTX (30 μg) and CAZ (30 μg) disks in combination with 10 μg clavulanate (CLA) were performed and interpreted using NCCLS criteria for ESBL screening and disk confirmation tests. Disks for_ESBL confirmation tests were obtained from Oxoid Inc. (Nepean, Ontario, Canada). K. pneumoniae ATCC 700603 and E. coli ATCC 25922 were used as positive and negative controls, respectively.

Polymerase Chain Reaction (PCR) amplifications were carried out on a Thermal Cycler 9600 instrument (Applied Biosystems, Norwalk, Conn.) as indicated in Example 1, with the primers, the size of the expected amplification product, and annealing temperatures used for the PCR as listed in Table 1. Magnesium chloride concentrations were 1.5 mM for all PCR reactions.

TABLE 1
Control Strains producing well-characterized β-lactamases

| | | | CTX-M β- |
|---|---|---|---|
| | | | lactamase |
| Strain | Organism | β-lactamase | family Group |
| CF2 | E. cloacae | CTX-M-1 | I |
| Rio-4 | Proteus mirabilis | CTX-M-2 | II |
| VER-1 | E. cloacae | CTX-M-3 | I |
| Cfr2525/96 | Citrobacter freundii | CTX-M-3 | I |
| Eco3553/98 | E. coli | CTX-M-15 | I |
| 34 | Salmonella typhimurium | CTX-M-5 | II |
| Rio-3 | E. aerogenes | CTX-M-8 | III |
| 785D | E. coli | CTX-M-9 | IV |
| EC97/38582 | E. coli | CTX-M-10 | I |
| EC984167 | E. coli | CTX-M-14 | IV |
| CF1 | E. coli | CTX-M-14 | IV |
| Rio-6 | E. coli | CTX-M-16 | IV |
| BM4493 | K. pneumoniae | CTX-M-17 | IV |
| ILT-2 | K. pneumoniae | CTX-M-18 | IV |
| ILT-3 | K. pneumoniae | CTX-M-19 | IV |
| CT1* | E. coli | TOHO-l | II |
| S1* | E. coli | SHV-2 | — |
| S2* | E. coli | SHV-7 | — |
| T1* | E. coli | TEM-3 | — |
| T4* | E. coli | TEM-10 | — |
| T5* | E. coli | TEM-50 | — |

*These strains are part of an unpublished isogenic panel.

Results:

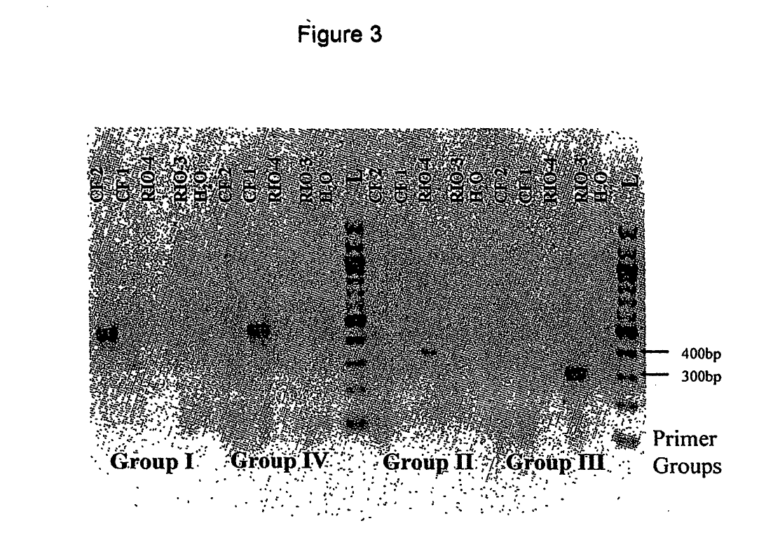

The designed primers were tested for specificity in separate PCR reactions using DNA template prepared from control strains known to produce specific CTX-M β-lactamases or strains producing ESBLs other than CTX-M β-lactamases (Tables 1 and 2, FIG. 3). PCR amplification of DNA template prepared from strains CF2, VER-1, Cfr2525/96, Eco3553/98, and EC97/38582, resulted in a single amplified product of 499 base pairs (bp) when CTX-M group I primers were used. No amplified product was identified with this primer set when DNA template from the rest of the control strains in Table 1 were used during PCR amplification. Group II primers amplified a single 351 bp fragment when DNA template was prepared from control strains Rio-4, 34, and CT1. All other control strain DNA templates resulted in no amplification product for this primer set.

TABLE 2
Laboratory diagnosis of control strains producing known extended-spectrum β-lactamases

| | | | | | Disk | | PCR primer pairs | | | |
|---|---|---|---|---|---|---|---|---|---|---|
| | | Screening^{a} | | | Confirmation^{b} | | CTX-M-I | CTX-M-II | CTX-M-III | CTX-M-VI |
| Strain | Enzyme | CPD | CAZ | CTX | CAZ/CL | CTX/CL | group | group | group | group |
| CF2 | CTX-M-1 | + | + | + | − | + | + | − | − | − |
| Rio-4 | CTX-M-2 | + | − | + | − | + | − | + | − | − |
| VER-1 | CTX-M-3 | + | + | + | − | + | + | − | − | − |
| Cfr2525/96 | CTX-M-3 | + | + | + | − | + | + | − | − | − |
| Eco3553/98 | CTX-M-15 | + | + | + | + | + | + | − | − | − |
| 34 | CTX-M-5 | + | + | + | − | + | − | + | − | − |
| Rio-3 | CTX-M-8 | + | − | + | − | + | − | − | + | − |
| 785D | CTX-M-9 | + | − | + | − | + | − | − | − | + |
| EC/9738582 | CTX-M-10 | + | − | + | − | + | + | − | − | − |
| EC984167 | CTX-M-14 | + | − | + | − | + | − | − | − | + |
| CF1 | CTX-M-14 | + | − | + | − | + | − | − | − | + |
| Rio-6 | CTX-M-16 | + | + | + | + | + | − | − | − | + |
| BM4493 | CTX-M-17 | + | + | + | + | + | − | − | − | + |
| ILT-2 | CTX-M-18 | + | + | + | − | + | − | − | − | + |
| ILT-3 | CTX-M-19 | + | + | + | + | + | − | − | − | + |
| CT1 | TOHO-1 | + | − | + | − | + | − | + | − | − |
| S1 | SHV-2 | + | + | + | + | + | − | − | − | − |
| S2 | SHV-7 | + | + | + | + | + | − | − | − | − |
| T1 | TEM-3 | + | + | + | + | + | − | − | − | − |
| T4 | TEM-10 | + | + | − | + | + | − | − | − | − |
| T5 | TEM-50 | + | + | + | + | + | − | − | − | − |

^{a}NCCLS guidelines for screening ESBL-producing bacteria. CPD: cefpodoxime (1 μg/ml); CAZ: ceftazidime (1 μg/ml); CTX: cefotaxime (1 μg/ml).
^{b}NCCLS guidelines for ESBL disk confirmation tests: CAZ: ceftazidime; CTX: cefotaxime; CLA: clavulanic acid.

Group IV primers amplified a 474 bp product from DNA prepared from strains 785D, EC984167, CF1, Rio-6, BM4493, ILT-2, and ILT-3. This primer set was also very specific resulting in no amplification of DNA when template was prepared from the other control strains. Using the group III primer set resulted in amplification of DNA prepared from only one strain, Rio-3, which produced CTX-M-8. An isolate producing CTX-M-25 was requested but not obtained. Therefore, we were unable to examine the ability of the Group III primer set to amplify blaCTX-M-25. A representative gel indicating the specificity of the primer pairs is shown in FIG. 2. These data indicate a high level of specificity for these group-specific primer pairs.

All the ESBL-producing E. coli and Klebsiella spp. were examined by PCR for the presence of blaCTX-M genes. Of the 168 E. coli isolated during the study period, 24 (14 %) were positive for blaCTX-M genes from the CTX-M-I group, indicating CTX-M-1-like β-lactamases. Ninety-three (55%) were positive for blaCTX-M genes from the CTX-M-IV group indicating CTX-M-14-like β-lactamases and the remaining 51 (31%) were negative for blaCTX-M genes (Table 4). Of the 7 K. pneumoniae isolated during the study period, 2 (29%) were positive for blaCTX-M genes from the CTX-M-IV group and the remaining 5 (71 %) were negative for blaCTX-M genes (Table 3).

TABLE 3
Laboratory diagnosis of clinical strains producing extended-spectrum β-lactamases

| | | | | | | | | Disk confirmation^{b} | | | | | |
|---|---|---|---|---|---|---|---|---|---|---|---|---|---|
| | | Screening^{a} | | | | | | No positive (%) | | | | | |
| | PCR for bla_{CTXM} | No positive (%) | | | | | | | | | | CTX/CLA | |
| Strains | genes^{c} | CPD | | CAZ | | CTX | | CAZ/CLA | | CTX/CLA | | and CAZ/CLA | |
| E. coli | Negative | 51 | (100%) | 51 | (100%) | 48 | (94%) | 34 | (67%) | 49 | (96%) | 51 | (100%) |
| (n = 51) | | | | | | | | | | | | | |
| K. pneumoniae | Negative | 5 | (100%) | 5 | (100%) | 4 | (80%) | 3 | (60%) | 5 | (100%) | 5 | (100%) |
| (n = 5) | | | | | | | | | | | | | |
| E. coli | Positive for | 24 | (100%) | 24 | (100%) | 24 | (100%) | 21 | (88%) | 24 | (100%) | 24 | (100%) |
| (n = 24) | CTX-M-1 group | | | | | | | | | | | | |
| E. coli | Positive for | 93 | (100%) | 71 | (76%) | 93 | (100%) | 3 | (3%) | 93 | (100%) | 93 | (100%) |
| (n = 93) | CTX-M-1V group | | | | | | | | | | | | |
| K. pneumoniae | Positive for | 2 | (100%) | 0 | (0%) | 2 | (100%) | 0 | (0%) | 2 | (100%) | 2 | (100%) |
| (n = 2) | CTX-M-1V group | | | | | | | | | | | | |
| Total | | 175 | (100%) | 151 | (86%) | 171 | (98%) | 61 | (35%) | 173 | (99%) | 175 | (100%) |
| (n = 175) | | | | | | | | | | | | | |

^{a}NCCLS guidelines for screening ESBL-producing bacteria. CPD: cefpodoxime (1 μg/ml); CAZ: ceftazidime (1 μg/ml); CTX: cefotaxime (1 μg/ml). Number of strains of positive (percentage %).
^{b}NCCLS guidelines for ESBL disk confirmation tests: CAZ: ceftazidime; CTX: cefotaxime; CLA: clavulanic acid.
^{c}Group I includes CTX-M-1, 3, 10-12, 15 (UOE-1), 22, 23, 28, 29, 30; Group II includes CTX-M-2, 4-7, 20, and TOHO-1; Group III includes CTX-M-8; Group IV includes CTX-M-9, 13, 14, 16-19, 21, 27, and TOHO-2; and Group V includes CTX-M-25 and 26.

### EXAMPLE 3

### Population-Based Surveillance for ESBL-Producing E. coli Infections

Methods:

A total of 157 isolates were collected from a population-based surveillance of ESBL-producing E. coli infections. The E. coli was isolated by standard techniques, and susceptibilities to antimicrobial agents were determined using Vitek AMS (bioMérieux Vitek Systems). The presence of an ESBL was established on the basis of NCCLS guidelines. All strains with an MIC of cefpodoxime of ≧8 μg/mL were subjected to the NCCLS disk confirmation tests by cefotaxime (CTX; 30 μg) and ceftazidime (CAZ; 30 μg) disks in combination with 10 μg of clavulanate (CLA). The results were interpreted using NCCLS criteria.

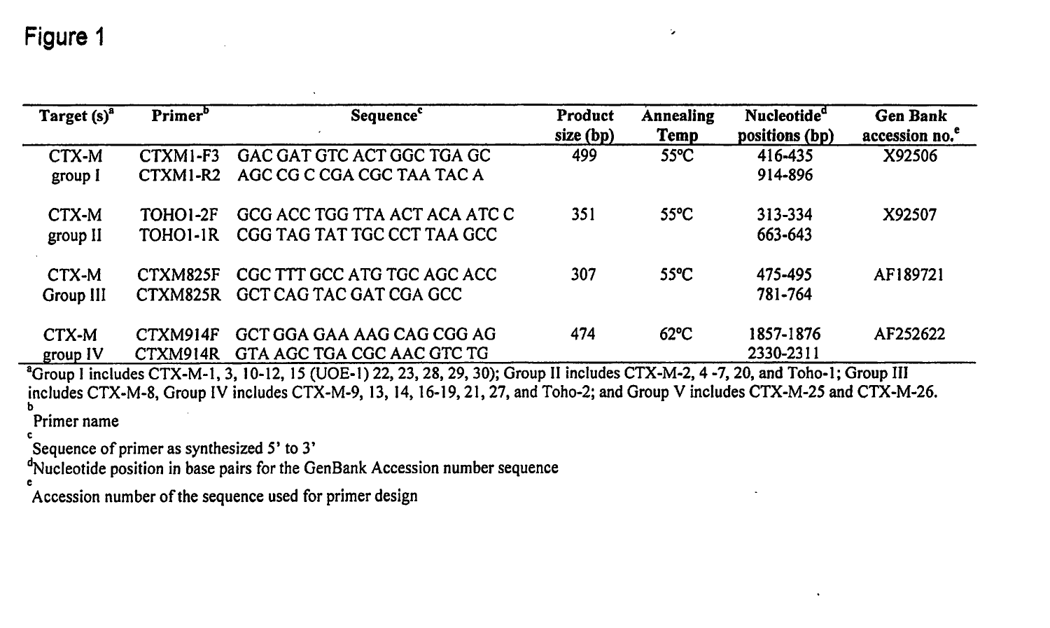

PCR amplification for CTX-M β-lactamase genes was performed using a Thermal Cycler 9600 instrument (Applied Biosystems, Norwalk, Conn.) with standard PCR conditions, as described in Example 1 using primer pairs for CTX-M Groups 1-5 β-lactamases, set forth as SEQ ID NO. 1 through SEQ ID NO. 8 indicated above and in FIG. 1.

All analyses were performed using Stata statistical software, version 8.0 (StataCorp, College Station, Tex.). Differences in proportions among categorical data were assessed using Fisher's exact test. Incidence rates were calculated by using regional demographic data as the denominator and compared with Poisson counts. Category-specific risks were calculated and reported as relative risk (RR) with 95% CIs, as described in Laupland et al., J. Infect. Dis., 187:1452-1459 (2003). A multivariable logistic regression model was developed to assess factors associated with the presence of bla_{CTX-M} genes. Variables in the initial model included all of those significant to in P<0.1 univariate analysis, age, sex, and community/nosocomial onset. Backward stepwise variable elimination was performed to derive the final model. Calibration and discrimination were assessed using the Hosmer-Lemeshow goodness-of-fit test and the area under the receiver operator curve (ROC), respectively. Results were reported as ORs and 95% CIs.

Results:

All ESBL-producing E. coli were examined by PCR for the presence of bla_{CTX-M} genes. Twenty-three (15%) of 157 were positive for bla_{CTX-M} genes from the CTX-M-I subgroup (referred to as “CTX-M-1-like β-lactamases), 87 (55%) were positive for bla_{CTX-M} genes from CTX-M-III subgroup (CTX-M-14-like β-lactamases), and the remaining 47 (30%) were negative for _{blaCTX-M} genes (CTX-M negative).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A primer selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof.

2. A primer pair comprising one primer selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof.

3. A primer selected from the group consisting of:
5′-GAC GAT GTC ACT GGC TGA GC-3′(SEQ ID NO:1); and
5′-AGC CGC CGA CGC TAA TAC A-3′(SEQ ID NO:2); and full-length complements thereof.

4. A primer selected from the group consisting of:
5′-GCG ACC TGG TTA ACT ACA ATC C-3′(SEQ ID NO:3); and
5′-CGG TAG TAT TGC CCT TAA GCC-3′(SEQ ID NO:4); and full-length complements thereof.

5. A primer selected from the group consisting of:
5′-GCT GGA GAA AAG CAG CGG AG-3′(SEQ ID NO:5); and
5′-GTA AGC TGA CGC AAC GTC TG-3′(SEQ ID NO:6); and full-length complements thereof.

6. A primer selected from the group consisting of:
5′-CGC TTT GCC ATG TGC AGC ACC-3′(SEQ ID NO:7); and
5′-GCT CAG TAC GAT CGA GCC-3′(SEQ ID NO:8); and full-length complements thereof.

7. A method for identifying a β-lactamase in a clinical sample, the method comprising:
providing a pair of oligonucleotide primers specific for a nucleic acid specific for 1 or more groups of the CTX-M β-lactamase family, wherein one primer of the pair is complementary to at least a portion of the β-lactamase nucleic acid in the sense strand and the other primer of each pair is complementary to at least a portion of the β-lactamase nucleic acid in the antisense strand;
annealing the primers to the β-lactamase nucleic acid;
simultaneously extending the annealed primers from a 3′ terminus of each primer to synthesize an extension product that is complementary to the nucleic acid strands annealed to each primer wherein each extension product after separation from the β-lactamase nucleic acid serves as a template for the synthesis of an extension product for the other primer of each pair;
separating the amplified products; and
analyzing the separated amplified products for a region characteristic of the β-lactamase.

8. The method of claim 7 wherein analyzing the separated amplified products comprises visual inspection of a gel obtained from a gel electrophoresis of the separated amplified products.

9. The method of claim 8 further comprising at least one additional assay to provide further identification of the β-lactamase in the clinical sample.

10. The method of claim 9 wherein the at lest one additional assay is selected from the group consisting of restriction fragment length polymorphism (RFLP), WAVE analysis, sequence identification (Gold Standard), single stranded conformational polymorphisms (SSCP), and combinations thereof.

11. The method of claim 7 wherein the pair of oligonucleotide primers are specific for a nucleic acid characteristic of one group of the CTX-M β-lactamase family.

12. The method of claim 7 wherein the pair of oligonucleotide primers are specific for a nucleic acid characteristic of two groups of the CTX-M β-lactamase family.

13. The method of claim 7 wherein the primers are specific for a nucleic acid characteristic of at least one β-lactamase enzyme selected from the group consisting of CTX-M 1, 2, 3, 4, 5, 6, 7, 8, 9, 10, 11, 12, 13, 14, 15 (UOE-1), 16, 17, 18, 19, 20, 21, 22, 23, 25, 26, 27, 28, 29, 30, TOHO-1, TOHO-2, and combinations thereof.

14. The method of claim 7 wherein the primers are selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof.

15. The method of claim 7 wherein the pair of oligonucleotide primers is specific for at least one nucleic acid characteristic of the CTX-M-1, 3, 10-12, 15 (UOE-1), 22, 23, 28, 29 and 30 β-lactamase enzymes.

16. The method of claim 15 wherein the primers are selected from the group consisting of:
5′-GAC GAT GTC ACT GGC TGA GC-3′(SEQ ID NO:1); and
5′-AGC CGC CGA CGC TAA TAC A-3′(SEQ ID NO:2); and full-length complements thereof.

17. The method of claim 7 wherein the pair of oligonucleotide primers is specific for at least one nucleic acid characteristic of the CTX-M-2, 4, 5, 6, 7, 20, and TOHO-1 β-lactamase enzymes.

18. The method of claim 17 wherein the primers are selected from the group consisting of:
5′-GCG ACC TGG TTA ACT ACA ATC C-3′(SEQ ID NO:3); and
5′-CGG TAG TAT TGC CCT TAA GCC-3′(SEQ ID NO:4); and full-length complements thereof.

19. The method of claim 7 wherein the pair of oligonucleotide primers is specific for at least one nucleic acid characteristic of the CTX-M-9, 13, 14, 16, 17, 18, 19, 21, 27, and TOHO-2 β-lactamase enzymes.

20. The method of claim 19 wherein the primers are selected from the group consisting of:
5′-GCT GGA GAA AAG CAG CGG AG-3′(SEQ ID NO:5); and
5′-GTA AGC TGA CGC AAC GTC TG-3′(SEQ ID NO:6); and full-length complements thereof.

21. The method of claim 7 wherein the pair of oligonucleotide primers is specific for at least one nucleic acid characteristic of the CTX-M-8, 25 and 26 β-lactamase enzymes.

22. The method of claim 21 wherein the primers are selected from the group consisting of:
5′-CGC TTT GCC ATG TGC AGC ACC-3′(SEQ ID NO:7); and
5′-GCT CAG TAC GAT CGA GCC-3′(SEQ ID NO:8); and full-length complements thereof.

23. A method for identifying a β-lactamase in a clinical sample, the method comprising:
providing a primer pair comprising one primer selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof; and
subjecting the primer pair to a real-time polymerase chain reaction assay.

24. A diagnostic kit for detecting a CTX-M β-lactamase which comprises packaging, containing, separately packaged:
(a) at least one primer pair capable of hybridizing to a β-lactamase nucleic acid selected from the group consisting of members of Groups 1-5 of the CTX-X β-lactamase family;
(b) a positive and negative control; and
(c) a protocol for identification of the β-lactamase nucleic acid of interest;
wherein the primer pair is specific for one or more groups within the CTX-M β-lactamase family.

25. The diagnostic kit of claim 24 wherein the primers are selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof.

26. A diagnostic kit for detecting a CTX-M β-lactamase which comprises packaging, containing, separately packaged:
(a) at least one primer pair capable of hybridizing to a β-lactamase nucleic acid of interest;
(b) a positive and negative control; and
(c) a protocol for identification of the β-lactamase nucleic acid of interest;
wherein the primers are selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof.

27. A diagnostic kit for detecting a CTX-M β-lactamase using real-time polymerase chain reaction which comprises packaging, containing, separately packaged:
(a) at least one primer pair capable of hybridizing to a β-lactamase nucleic acid of interest;
(b) a positive and negative control; and
(c) a protocol for identification of the β-lactamase nucleic acid of interest;
wherein one primer of the primer pair is selected from the group consisting of:
5′-GAC GAT GTC ACT GGC TGA GC-3′(SEQ ID NO:1);
5′-AGC CGC CGA CGC TAA TAC A-3′(SEQ ID NO:2);
5′-GCG ACC TGG TTA ACT ACA ATC C-3′(SEQ ID NO:3);
5′-CGG TAG TAT TGC CCT TAA GCC-3′(SEQ ID NO:4);
5′-GCT GGA GAA AAG CAG CGG AG-3′(SEQ ID NO:5);
5′-GTA AGC TGA CGC AAC GTC TG-3′(SEQ ID NO:6);
5′-CGC TTT GCC ATG TGC AGC ACC-3′(SEQ ID NO:7); and
5′-GCT CAG TAC GAT CGA GCC-3′(SEQ ID NO:8); and full-length complements thereof.
